(19) United States
(12) Patent Application Publication
(10) Pub. No.: US 2025/0213809 A1
(43) Pub. Date: Jul. 3, 2025

(54) Ventilation System

(71) Applicants: University of Washington, Seattle, WA (US); Seattle Children's Hospital d/b/a/ Seattle Children’s Research Institute, Seattle, WA (US)

(72) Inventors: Amelie von Saint Andre-von Arnim, Seattle, WA (US); Robert Mariano Diblasi, Seattle, WA (US); Jonathan Arthur Poli, Seattle, WA (US); Ibukun Sonaike, Seattle, WA (US); Ofer Yanay, Seattle, WA (US); Andrew Vamos, Seattle, WA (US)

(21) Appl. No.: 18/852,188

(22) PCT Filed: Mar. 30, 2023
(86) PCT No.: PCT/US2023/065164

§ 371 Date: Sep. 27, 2024

Related U.S. Application Data
(60) Provisional application No. 63/325,976, filed on Mar. 31, 2022.

(51) Int. Cl. A61M 16/00

(52) U.S. Cl. CPC A61M 16/024; A61M 16/0003; A61M 2016/003; A61M 2202/0208; A61M 2205/3584; A61M 2205/505; A61M 2230/42

(57) ABSTRACT

A system includes a container configured to contain a liquid. The system also includes a tubing apparatus having a first port configured for connection to a gas source, a second port configured for connection to a patient interface, and a third port configured for submersion within the liquid. The system also includes an actuator configured to move the third port vertically within the container. A method includes flowing gas into the first port of the tubing apparatus to the patient interface via the second port of the tubing apparatus. The method also includes moving the third port of the tubing apparatus vertically within the container such that an amount of the liquid in the container that is above the third port changes, thereby changing a pressure at the patient interface.

### CROSS-REFERENCE TO RELATED APPLICATION

This application is an international application claiming priority to U.S. Provisional Application No. 63/325,976, filed on Mar. 31, 2022, the entire contents of which are incorporated by reference herein.

### BACKGROUND

Lower respiratory tract infections (LRTIs) are one of the leading causes of mortality in children worldwide. In addition to preventive measures, antibiotics, and supplemental oxygen therapy, further respiratory support is often needed for children with acute respiratory distress and failure secondary to an LRTI. Unfortunately, there is a limited availability of respiratory support devices in many areas.

### SUMMARY

A first example is a system comprising: a container configured to contain a liquid; a tubing apparatus having a first port configured for connection to a gas source, a second port configured for connection to a patient interface, and a third port configured for submersion within the liquid; and an actuator configured to move the third port vertically within the container.

A second example is a method comprising: flowing gas into a first port of a tubing apparatus to a patient interface via a second port of the tubing apparatus; and moving a third port of the tubing apparatus vertically within a container such that an amount of a liquid in the container that is above the third port changes, thereby changing a pressure at the patient interface.

A third example is a non-transitory computer readable medium storing instructions that, when executed by a computing device, cause the system of the first example to perform functions comprising flowing gas into a first port of a tubing apparatus to a patient interface via a second port of the tubing apparatus; and moving a third port of the tubing apparatus vertically within a container such that an amount of a liquid in the container that is above the third port changes, thereby changing a pressure at the patient interface.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/− 0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a block diagram of a computing device, according to an example.

FIG. 2 is a schematic diagram of a system, according to an example.

FIG. 3 is a block diagram of a method, according to an example.

FIG. 4 shows experimental data related to a system, according to an example.

FIG. 5 shows experimental data related to a system, according to an example.

FIG. 6 shows experimental data related to a system, according to an example.

FIG. 7 shows experimental data related to a system, according to an example.

FIG. 8 shows experimental data related to a system, according to an example.

FIG. 9 shows experimental data related to a system, according to an example.

FIG. 10 shows experimental data related to a system, according to an example.

FIG. 11 shows experimental data related to a system, according to an example.

### DETAILED DESCRIPTION

As noted above, there is a need for respiratory support solutions that are inexpensive and effective. This disclosure includes example systems and methods that can help satisfy this need.

A system includes a container such as a plastic bucket configured to contain a liquid such as water. The system further includes a tubing apparatus having a first port configured for connection to a gas source such as one or more pressurized gas tanks. The tubing apparatus also includes a second port configured for connection to a patient interface such as an oxygen mask. The tubing apparatus also includes a third port (e.g., an open end of a tube) configured for submersion within the liquid that is held by the container. The tubing apparatus can take the form of medical grade plastic tubing equipped with adapters or fittings that can be used to make airtight connections to the gas source at the first port and the patient interface at the second port. The system also includes an actuator such as an electric servomotor configured to move the third port vertically within the container and/or the liquid.

The system flows gas such as air or oxygen-enriched air into the first port of the tubing apparatus such that the gas flows through the tubing apparatus into the patient interface via the second port. This can assist the patient's breathing. Additionally, the actuator moves the third port of the tubing apparatus vertically within the container such that an amount (e.g., a depth) of the liquid in the container that is above the third port changes, thereby changing a pressure at the patient interface. For example, when the actuator lowers the third port deeper into the liquid, the pressure at the patient interface increases. Likewise, when the actuator raises the third port to a shallower position within the liquid, the pressure at the patient interface decreases. In some examples, the higher pressure at the patient interface caused by the third port being positioned deep within the liquid can assist the patient with inhalation, whereas the lower pressure at the patient interface caused by the third port being positioned shallow within the liquid can facilitate easier exhalation.

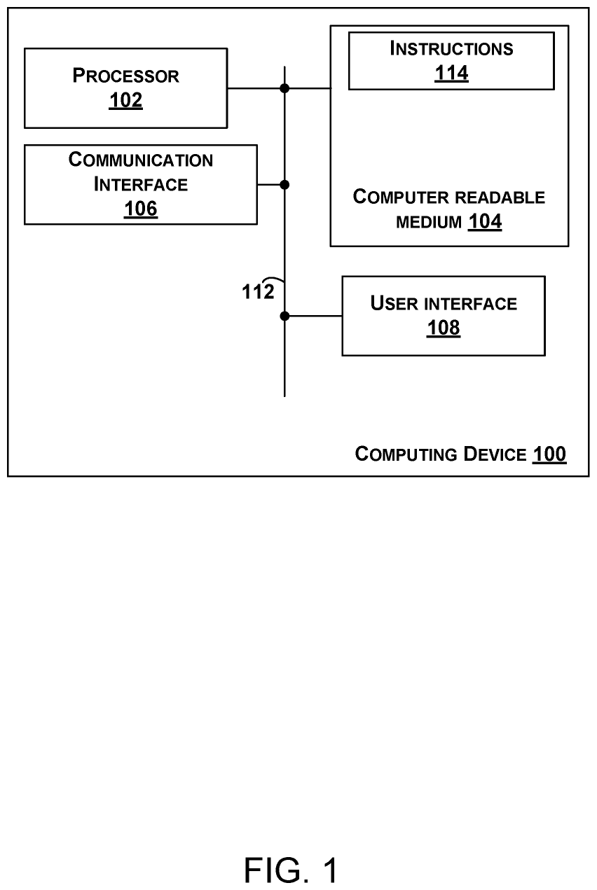

FIG. 1 is a block diagram of a computing device 100. The computing device 100 includes one or more processors 102, a non-transitory computer readable medium 104, a communication interface 106, and a user interface 108. Components of the computing device 100 are linked together by a system bus, network, or other connection mechanism 112.

The one or more processors 102 can be any type of processor(s), such as a microprocessor, a field programmable gate array, a digital signal processor, a multicore processor, etc., coupled to the non-transitory computer readable medium 104.

The non-transitory computer readable medium 104 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 104 can store instructions 114. The instructions 114 are executable by the one or more processors 102 to cause the computing device 100 to perform any of the functions or methods described herein.

The communication interface 106 can include hardware to enable communication within the computing device 100 and/or between the computing device 100 and one or more other devices. The hardware can include any type of input and/or output interfaces, a universal serial bus (USB), PCI Express, transmitters, receivers, and antennas, for example. The communication interface 106 can be configured to facilitate communication with one or more other devices, in accordance with one or more wired or wireless communication protocols. For example, the communication interface 106 can be configured to facilitate wireless data communication for the computing device 100 according to one or more wireless communication standards, such as one or more Institute of Electrical and Electronics Engineers (IEEE) 801.11 standards, ZigBee standards, Bluetooth standards, etc. As another example, the communication interface 106 can be configured to facilitate wired data communication with one or more other devices. The communication interface 106 can also include analog-to-digital converters (ADCs) or digital-to-analog converters (DACs) that the computing device 100 can use to control various components of the computing device 100 or external devices.

The user interface 108 can include any type of display component configured to display data. As one example, the user interface 108 can include a touchscreen display. As another example, the user interface 108 can include a flat-panel display, such as a liquid-crystal display (LCD) or a light-emitting diode (LED) display. The user interface 108 can include one or more pieces of hardware used to provide data and control signals to the computing device 100. For instance, the user interface 108 can include a mouse or a pointing device, a keyboard or a keypad, a microphone, a touchpad, or a touchscreen, among other possible types of user input devices. Generally, the user interface 108 can enable an operator to interact with a graphical user interface (GUI) provided by the computing device 100 (e.g., displayed by the user interface 108).

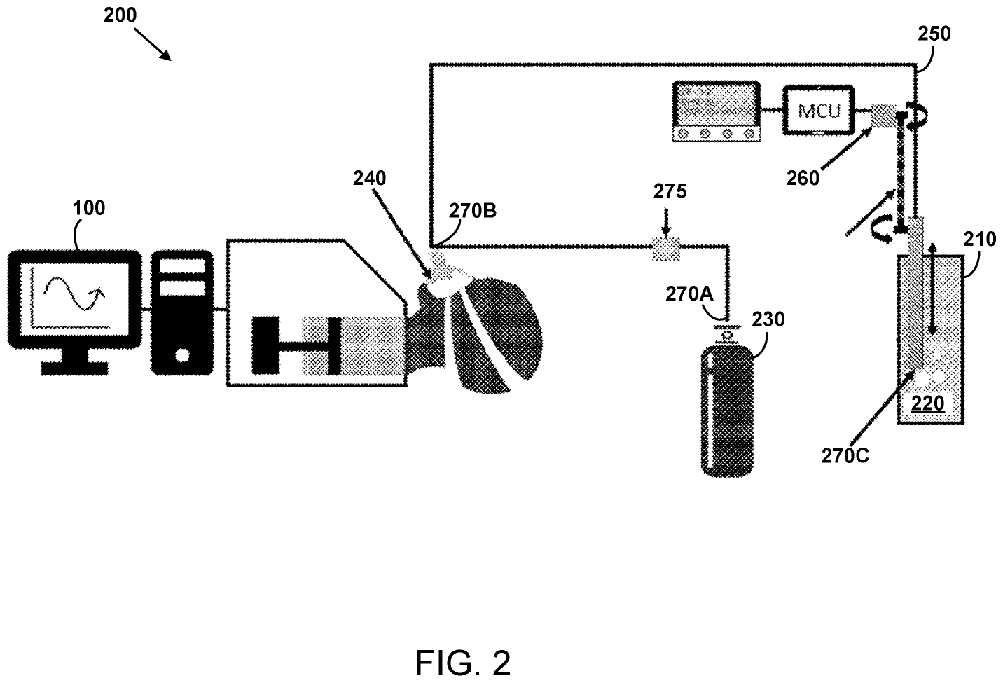

FIG. 2 is a schematic diagram of a system 200. The system 200 includes the computing device 100, a container 210 that contains a liquid 220, a gas source 230, a patient interface 240, a tubing apparatus 250, an actuator 260, and a flow sensor 275. The tubing apparatus 250 includes a port 270A connected to the gas source 230, a port 270B connected to the patient interface 240, and a port 270C submersed within the liquid 220 that is held by the container 210.

The container 210 typically takes the form of a plastic bucket with an open upper end through which the port 270C of the tubing apparatus 250 is inserted.

The liquid 220 generally takes the form of water.

The gas source 230 typically includes one or more pressurized gas tanks containing air, oxygen, and/or oxygen-enriched air. The flow sensor 275 is used to control (e.g., via the computing device 100) an amount of gas that is provided by the gas source 230 to the patient interface 240 via the port 270A and the port 270B. The gas source 230 is configured to generate a gradient of decreasing pressure within the tubing apparatus 250 from the gas source 230 to the patient interface 240. That is, the pressure within the tubing apparatus 250 decreases from the port 270A to the port 270B.

The patient interface 240 generally takes the form of a plastic oxygen mask.

The tubing apparatus 250 typically includes one or more pieces of medical grade plastic tubing connected together with fittings and/or equipped with adapters that can be used to make airtight connections to the gas source 230 at the port 270A and the patient interface 240 at the port 270B. As such, the tubing apparatus 250 is configured to maintain a gradient of decreasing pressure within the tubing apparatus 250 from the port 270B to the port 270C. As shown, the port 270A is at a first end of the tubing apparatus 250, the port 270C is at a second end of the tubing apparatus 250 opposite the first end, and the port 270B is between the port 270A and the port 270C.

The actuator 260 is configured to move the port 270C vertically within the container 210. For example, the computing device 100 controls the actuator 260 to move the port 270C vertically within the container 210. The actuator 260 can include one or more of a linear actuator, a direct current motor, or a servomotor. Vertical movement of the port 270C within the liquid 220 changes the gradient of decreasing pressure that exists from the port 270B to the port 270C.

In operation, the system 200 flows gas from the gas source 230 into the port 270A to the patient interface 240 via the flow sensor 275 and the port 270B. The gas generally includes air, oxygen, and/or oxygen-enriched air.

Additionally, the system 200 moves, via the actuator 260, the port 270C vertically within the container 210 such that an amount of the liquid 220 in the container 210 that is above the port 270C changes, thereby changing a pressure at the patient interface 240 based on the amount of the liquid 220 in the container 210 that is above the port 270C.

In some examples, the system 200 moves the port 270C periodically and vertically from a first position within the liquid 220 to a second position within the liquid 220 and back to the first position. In this example, the first position is below (e.g., deeper within the liquid 220 than) the second position. The port 270C being at the first position can coincide with the patient inhaling and the port 270C being at the second position can coincide with the patient exhaling.

In some examples, the system 200 can be used to control and assist the patient's respiration cycle. For example, the system 200 receives a selection of a respiratory rate (e.g., 0.25 Hz) via the user interface 108 of the computing device 100. In response, the system 200 moves the port 270C from the first position to the second position and back to the first position periodically according to the selected respiratory rate.

In a similar manner, the system 200 receives a selection of an inspiratory time (e.g., 1.5 seconds) via the user interface 108. In response, the system 200 moves the port 270C such that, for each respiration cycle, the port 270C is at the first position for a time equal to the inspiratory time.

Likewise, the system 200 receives a selection of an expiratory time (e.g., 1.5 seconds) via the user interface 108. In response, the system 200 moves the port 270C such that, for each respiration cycle, the port 270C is at the second position for a time equal to the expiratory time.

Additionally or alternatively, the system 200 receives a selection of an inspiration pressure via the user interface 108. In response, the system 200 moves the port 270C to the first position to generate a pressure at the patient interface 240 that is substantially equal to the selected inspiration pressure.

Additionally or alternatively, the system 200 receives a selection of an expiration pressure via the user interface 108. In response, the system 200 moves the port 270C to the second position to generate a pressure at the patient interface 240 that is substantially equal to the selected expiration pressure.

In other examples, the system 200 conforms its operation cycle to the patient's natural respiratory cycle instead of controlling the timing of the patient's respiratory cycle. As such, the system 200 senses, via a sensor such as a pressure sensor located at the port 270B, a respiratory rate of the patient. In response, the system 200 moves the port 270C within the liquid 220 back and forth between the first position and the second position periodically according to the sensed respiratory rate.

Typically, the system 200 and the actuator 260 use closed loop feedback to control the position of the port 270C. That is, the actuator 260 can take the form of a servomotor that has a position encoder that generates output indicative of the actual position of the port 270C. Thus, the system 200 senses the vertical position of the port 270C within the liquid 220 and adjusts a force applied to the port 270C based on the sensed vertical position. For example, the actuator 260 can increase the force applied to the port 270C if the port 270C is trailing the expected position of the port 270C and can decrease the force applied to the port 270C if the port 270C is leading the expected position of the port 270C.

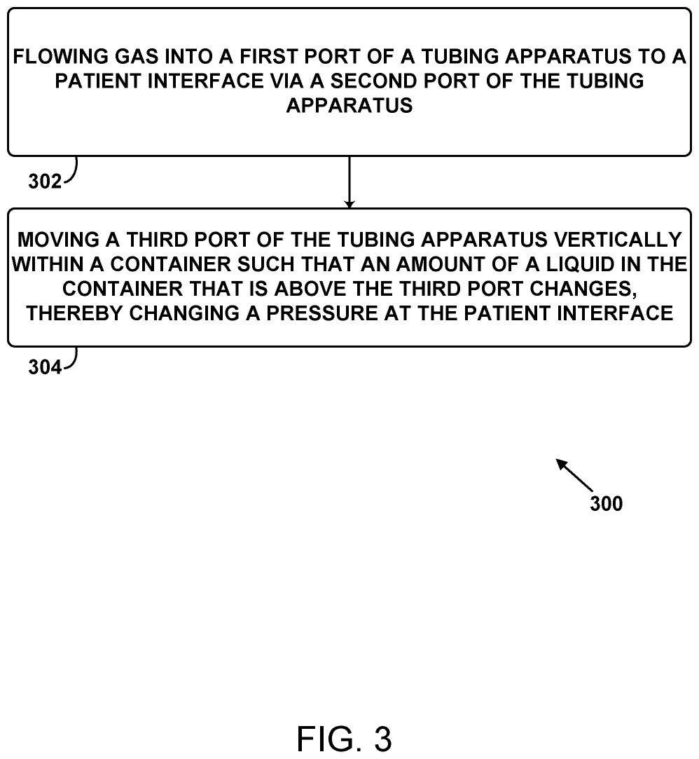

FIG. 3 is a block diagram of a method 300, which in some examples is performed by the system 200. As shown in FIG. 3, the method 300 includes one or more operations, functions, or actions as illustrated by blocks 302 and 304. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 302, the method 300 includes flowing gas into the port 270A of the tubing apparatus 250 to the patient interface 240 via the port 270B of the tubing apparatus 250. Functionality related to block 302 is described above with reference to FIG. 2.

At block 304, the method 300 includes moving the port 270C of the tubing apparatus 250 vertically within the container 210 such that an amount of the liquid 220 in the container 210 that is above the port 270C changes, thereby changing a pressure at the patient interface 240. Functionality related to block 304 is described above with reference to FIG. 2.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

### Further Examples

Bubble continuous positive airway pressure (BCPAP) is a respiratory support technique utilizing continuous gas flow in a column of water to create airway pressures that are adjustable by the depth of the expiratory tubing under water. BCPAP can be more effective than ventilator continuous positive airway pressure (CPAP) based on airway pressure vibrations from water bubbles thought to contribute to gas exchange and lung recruitment. In neonates with respiratory distress in low or middle income countries (LMICs), BCPAP reduced the need for mechanical ventilation by 30-50% and it is generally favored because of its perceived safety, non-invasiveness, limited technical skill requirements, and low cost compared with mechanical ventilation. Two randomized, controlled trials in Bangladesh and Ghana showed physiologic and mortality benefits with the use of BCPAP in select pediatric populations. Sicker children may need respiratory support beyond CPAP, including nasal intermittent positive pressure ventilation (NIPPV) or invasive mechanical ventilation to support respiratory failure. Although NIPPV is non-invasive and therefore potentially easier and safer to implement in LMICs than invasive ventilation, both require dedicated ventilators which are neither readily affordable nor available in low resource settings.

A simple, low-cost dual pressure device based on a BCPAP modification was developed for the neonatal population and produced pressure waveforms and tidal volumes comparable to commercial ventilators at commonly used neonatal pressures in a non-invasive mechanical lung and invasively ventilated animal model. However, this device lacks sensing and controls for inspiratory and expiratory times with risk for dysynchrony between patient respiratory effort and controlled ventilator breaths.

Therefore, a low cost, non-invasive, dual pressure respiratory support device adaptation based on BCPAP technology, targeting a wider pediatric age range and with additional controls for respiratory cycle may be desirable.

The present disclosure provides a low-cost bubble PAP device, utilizing hydrostatic depth pressures that will provide CPAP, NIPPV, and invasive ventilation similar to standard NIPPV and ventilator machines. Inspiratory and expiratory pressures are generated by a vertically moving tube submerged in a tank of water at timed intervals. The system can include a motor that drives tubing vertically into a water container at varying depths, providing dual pressure.

For the inspiratory limb, the patient is connected to an air source and flow sensor. The expiratory limb containing the bubble PAP device consists of a dynamic expiratory tube submerged in a water tank. CPAP is provided when the tube is static in the water column. A microcontroller and motor are configured to intermittently adjust the tube height to achieve dual pressure (CPAP and peak inspiratory pressure) respiratory support with various parameters (inspiratory and expiratory times, respiratory rate, positive inspiratory pressure (PIP), and positive end expiratory pressure (PEEP)) adjustable by the operator.

In one example, attached to the tube is a GT2 belt connected to a direct current motor powered by a motor driver. The tube and belt can be mounted vertically using custom 3D-printed parts and GT2 pulleys so that any rotational motor movement results in linear actuation of the tube to variable submersion depths within the water tank. In one example, the motor is configured to rapidly fluctuate between two set points serving as PIP and PEEP levels. This was controlled by the microcontroller unit (MCU) programmed with a proportional integral derivative controller while the motor encoder is monitored via MCU interrupts. This motor control scheme allows for accurate and precise tube positioning. The settings for PIP, PEEP, breaths per minute (respiratory rate), and inspiratory and expiratory times were controllable by the operator via four rotary dials and displayed on a small LCD unit. The MCU and peripherals were powered with a 5V USB connection, and the motor driver was powered with a 12V DC source connected to wall power. Bias flow is provided via an air source with an adjustable flowrate valve and flow sensor.

Example systems include a container configured to contain water, a hollow elongated member having a first end and a second end opposite the first end, where the first end of the hollow elongated member is positioned within the container such that it is submerged in the water, a tube having a first end and a second end opposite the first end, where the first end of the tube is coupled to the second end of the hollow elongated member and the second end of the first tube is configured to be coupled to a patient interface, an actuator mechanism coupled to the hollow elongated member, where the actuator mechanism is configured to adjust a depth of the first end of the hollow elongated member within the container, and a user interface including a controller.

In one embodiment, the system comprises: (i) a container configured to contain water, (ii) a hollow elongated member having a first end and a second end opposite the first end, wherein the first end of the hollow elongated member is positioned within the container such that it is submerged in the water, (iii) a first tube having a first end and a second end opposite the first end, wherein the first end of the first tube is coupled to the second end of the hollow elongated member, and wherein the second end of the first tube is configured to be coupled to a patient interface, (iv) an actuator mechanism coupled to the hollow elongated member, wherein the actuator mechanism is configured to adjust a depth of the first end of the hollow elongated member within the container, and (v) a second tube having a first end and a second end opposite the first end, wherein the first end of the second tube is configured to be coupled to a pressurized air source, and wherein the second end of the second tube is configured to be coupled to the patient interface.

In another embodiment, the actuator mechanism comprises a linear actuator.

In another embodiment, the actuator mechanism comprises a direct current motor powered by a motor driver.

In another embodiment, the system further includes (i) a first pulley positioned adjacent and coupled to the direct current motor, (ii) a second pulley positioned adjacent the second end of the hollow elongated member, and (iii) a belt positioned around the first pulley and the second pulley and coupled to the hollow elongated member such that a rotation of the direct current motor is translated to a linear movement of the hollow elongated member.

In another embodiment, the actuator mechanism is configured to cause the hollow elongated member to fluctuate between two depths serving as positive inspiratory pressure (PIP) and positive end expiratory pressure (PEEP) levels.

In another embodiment, the system further includes (i) a controller in communication with the actuator mechanism, and (ii) a user interface in communication with the controller.

In another embodiment, the user interface includes settings for positive inspiratory pressure (PIP), positive end expiratory pressure (PEEP), breaths per minute, and inspiratory and expiratory times.

In another embodiment, the controller is in communication with one or more patient monitoring systems, and wherein the controller is configured to adjust one or more of the depth of the hollow elongated member and a pressure of air through the second tube in response to an indication of one or more parameters received from the one or more patient monitoring systems.

In another embodiment, the patient interface comprises a mask.

In another embodiment, the first tube provides a negative pressure to the patient interface to assists with an expiratory phase of breathing, and wherein the second tube provides a positive pressure to the patient interface to assist with an inspiratory phase of breathing.

An example includes a motor that drives tubing vertically into a water container at varying depths, providing dual pressure. The device was tested on a mechanical lung model (ASL 5000) simulating healthy lungs vs acute lung disease (ARDS) of a 20 kg child. Varying pressure targets and bias flows were used for proof of concept.

For the inspiratory limb, the ASL 5000 and airway models were connected to an air source and flow sensor. The expiratory limb contained the bubble BiPAP device consisting of the dynamic expiratory tube submerged in a water tank. A microcontroller and motor rapidly adjusted the tube height to achieve dual pressure respiratory support with various parameters (inspiratory and expiratory times, respiratory rate, PIP, PEEP) adjustable by the operator.

The bubble BiPAP device consisted of a dynamic expiratory tube linearly actuated via a custom jig while submerged in a water tank. Attached to the tube was a GT2 belt connected to a direct current motor (Motor #4841 25Dx63L mm-Pololu; Las Vegas, USA) powered by a motor driver (SHIELD-MDIO-Cytron Technologies; Penang, Malaysia). The tube and belt were mounted vertically using custom 3D-printed parts and GT2 pulleys so that any rotational motor movement resulted in linear actuation of the tube to variable submersion depths within the water tank. The motor rapidly fluctuated between two set points serving as positive inspiratory pressure (PIP) and positive end expiratory pressure (PEEP) levels. This was controlled by the microcontroller unit (MCU) (Elegoo Uno-Elegoo; Shenzhen, China) programmed with a proportional integral derivative controller operating near 40 Hz while the motor encoder was monitored via MCU interrupts. This motor control scheme allowed for accurate and precise tube positioning (generally +/−0.5 cm). The settings for PIP, PEEP, breaths per minute (respiratory rate), and inspiratory and expiratory times were controllable by the operator via four rotary dials and displayed on a small LCD unit. The MCU and peripherals were powered with a 5V USB connection, and the motor driver was powered with a 12V DC source connected to wall power. Bias flow was provided via an air source with an adjustable flowrate valve and flow sensor.

Breathing is simulated using age-specific normal and acute respiratory distress syndrome (ARDS) values in the ASL 5000 Test Lung (Ingmar Medical, Pittsburgh, Pennsylvania), a digitally controlled, high-fidelity breathing simulator, which utilizes mathematical modelling to simulate size and disease-specific pulmonary mechanics. Pulmonary pressures generated are tested repeatedly across different device settings and bias flows.

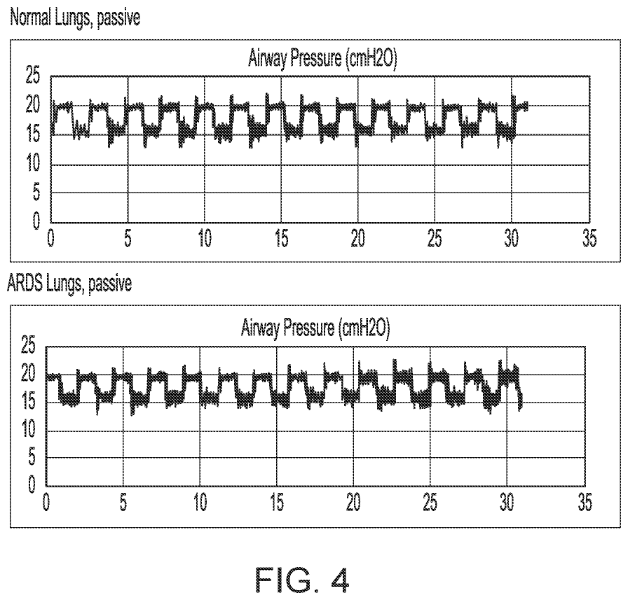

FIG. 4 shows the delivered pressure waveforms of a prototype. The high pressures (PIP) were set at 20 cmH₂O and low pressures (PEEP) set at 15 cmH₂O with a continuous source of air pressure (bias flow). The device was connected to ASL 5000 set at two different levels of lung compliance (healthy vs ARDS lungs). Each panel displays 30 seconds of pressure waveforms.

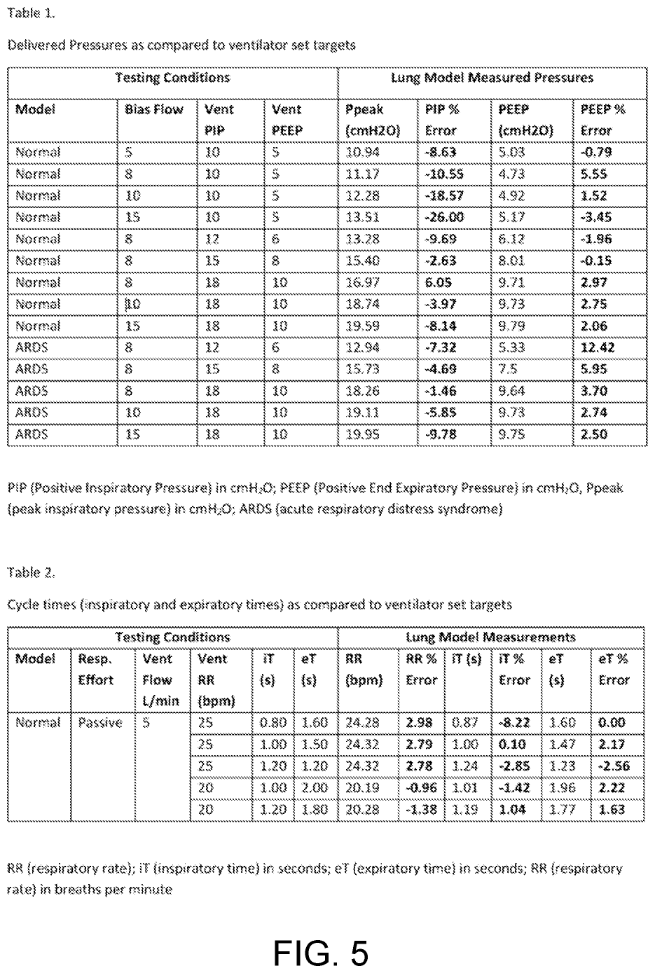

Table 1 shown in FIG. 5 contains measurements of pressures delivered to ASL 5000 Test Lung compared to pressure settings on bubble BiPAP (test lung Ppeak compared to set PIP and test lung PEEP compared to set PEEP) across different bias flows and lung compliance (healthy and ARDS lungs). PIP % Error and PEEP % Error are calculated percentages of how much pressures measured in the test lung differ from pressures set on bubble BiPAP.

Table 2 shown in FIG. 5 similarly contains inspiratory and expiratory times in seconds (iT and eT respectively), and respiratory rate measured in the test lung compared to same cycle times set on bubble BiPAP. iT % Error, eT % Error and RR % Error are calculated percentages of how much measured cycle times and respiratory differ from what was set in bubble BiPAP device. The maximum difference across these domains was less than +/−3%.

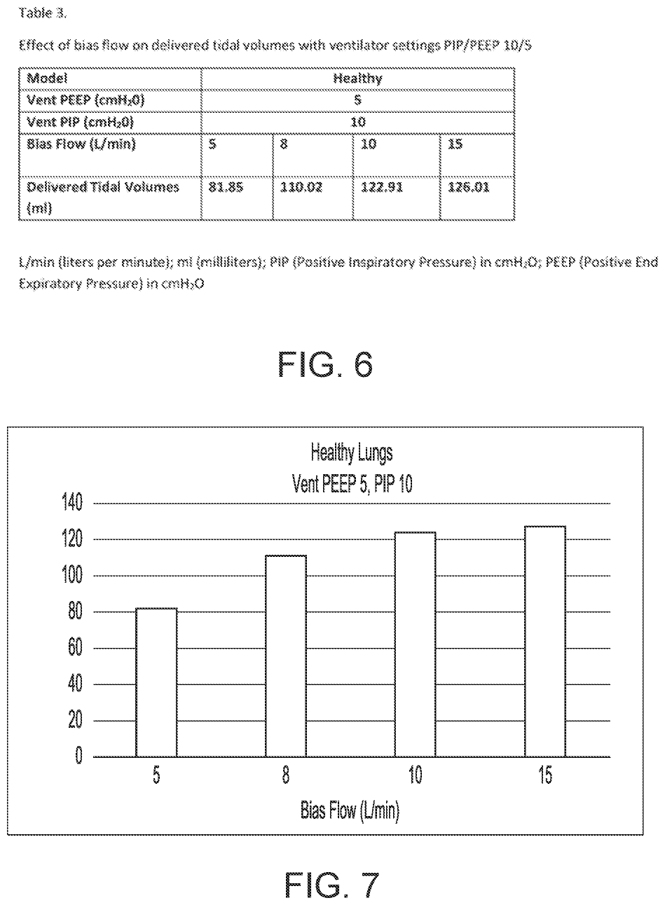

Table 3 shown in FIG. 6 and the bar graph of FIG. 7 show the effect of bias flows on measured tidal volumes (amount of air that moves in and out of lungs in each respiratory cycle in a healthy lung). The bubble device was set at PIP of 10 cmH₂O and PEEP of 5 cmH₂O. Tidal volumes increase with increased set bias flows. This is most notable on increasing flows from 5 L/min to 8 L/min.

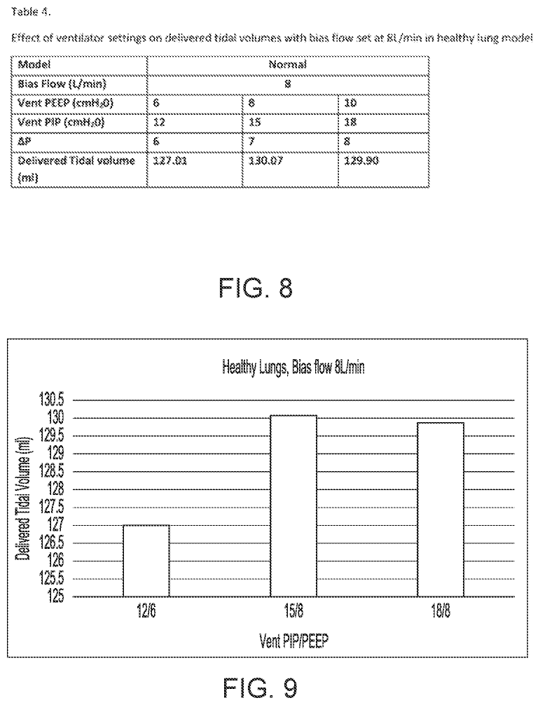

Table 4 shown in FIG. 8 and the bar graph of FIG. 9 show the effect of set pressures on measured tidal volumes using the same bias flow of 8 L/min in a healthy lung.

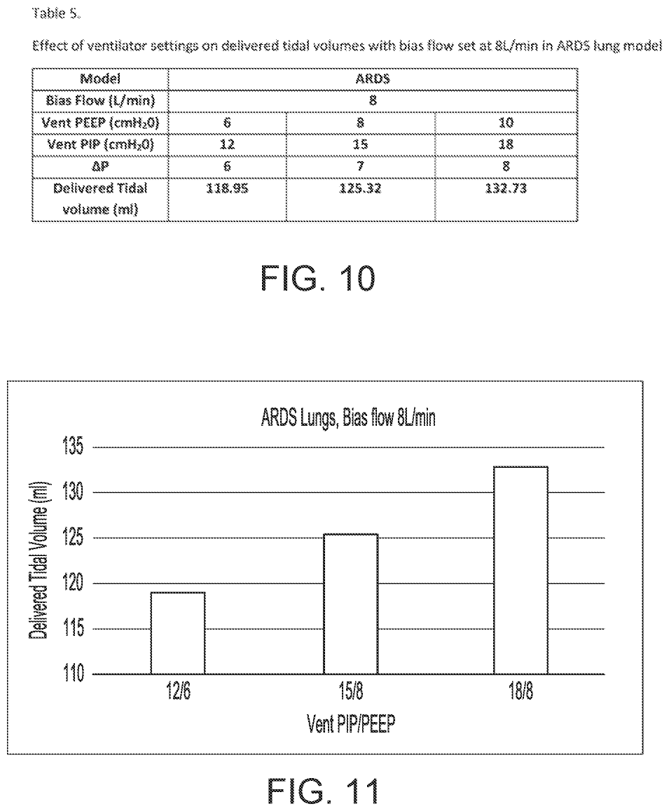

Table 5 shown in FIG. 10 and the bar graph of FIG. 11 show the effect of set pressures on measured tidal volumes using the same bias flow of 8 L/min with acute lung disease (ARDS). Both sets indicated increased measured tidal volumes with increased pressure settings on bubble BiPAP device. This effect is however blunted in the healthy lung model when pressures are increased to PIP/PEEP 18/10 cmH₂O from 15/8 cmH₂O.

1. A system comprising:
a container configured to contain a liquid;
a tubing apparatus having a first port configured for connection to a gas source, a second port configured for connection to a patient interface, and a third port configured for submersion within the liquid; and
an actuator configured to move the third port vertically within the container.

2. (canceled)

3. The system of claim 1, wherein the first port is at a first end of the tubing apparatus, the third port is at a second end of the tubing apparatus opposite the first end, and the second port is between the first port and the third port.

4-8. (canceled)

9. The system of claim 1, further comprising the gas source, wherein the gas source is configured to generate a gradient of decreasing pressure within the tubing apparatus from the gas source to the patient interface.

10. The system of claim 9, wherein the tubing apparatus is configured to maintain a second gradient of decreasing pressure within the tubing apparatus from the second port to the third port.

11. The system of claim 10, wherein moving the third port vertically within the liquid changes the second gradient of decreasing pressure.

12. The system of claim 1, further comprising a computing device configured to control an amount of gas flowing from the gas source through the tubing apparatus to the patient interface.

13. The system of claim 12, wherein the computing device is configured to control the actuator to move the third port vertically within the container.

14. A method comprising:
flowing gas into a first port of a tubing apparatus to a patient interface via a second port of the tubing apparatus; and
moving a third port of the tubing apparatus vertically within a container such that an amount of a liquid in the container that is above the third port changes, thereby changing a pressure at the patient interface.

15. The method of claim 14, wherein the first port is at a first end of the tubing apparatus, the third port is at a second end of the tubing apparatus opposite the first end, and the second port is between the first port and the third port.

16. The method of claim 14, wherein the flowing the gas comprises controlling a flow rate of the gas into the first port via a flow sensor that is between the first port and the second port.

17. The method of claim 14, wherein moving the third port comprises moving the third port periodically from a first position to a second position and back to the first position, wherein the first position is below the second position.

18. The method of claim 17, further comprising:
receiving a selection of a respiratory rate via a user interface,
wherein moving the third port periodically comprises moving the third port from the first position to the second position and back to the first position periodically according to the respiratory rate.

19. The method of claim 17, further comprising:
receiving a selection of an inspiratory time via a user interface,
wherein moving the third port periodically comprises moving the third port such that, for each respiration cycle, the third port is at the first position for a time equal to the inspiratory time.

20. The method of claim 17, further comprising:
receiving a selection of an expiratory time via a user interface,
wherein moving the third port periodically comprises moving the third port such that, for each respiration cycle, the third port is at the second position for a time equal to the expiratory time.

21. The method of claim 17, further comprising:
receiving a selection of an inspiration pressure via a user interface,
wherein moving the third port to the first position generates a pressure that is substantially equal to the inspiration pressure at the patient interface.

22. The method of claim 17, further comprising:
receiving a selection of an expiration pressure via a user interface,
wherein moving the third port to the second position generates a pressure that is substantially equal to the expiration pressure at the patient interface.

23. The method of claim 17, further comprising:
sensing, via a sensor at the second port, a respiratory rate of a patient,
wherein moving the third port periodically comprises moving the third port periodically according to the respiratory rate.

24. The method of claim 14, wherein moving the third port comprises sensing a vertical position of the third port and adjusting a force applied to the third port based on the vertical position.

25-28. (canceled)

29. The method of claim 14, wherein flowing the gas comprises flowing air, oxygen-enriched air, or oxygen to the patient interface while the patient interface is being worn by a patient.

30-31. (canceled)

32. A non-transitory computer readable medium storing instructions that, when executed by a computing device, cause the system of claim 1 to perform functions comprising:
flowing gas into the first port of the tubing apparatus to the patient interface via the second port of the tubing apparatus; and
moving the third port of the tubing apparatus vertically within the container such that an amount of the liquid in the container that is above the third port changes, thereby changing a pressure at the patient interface.

33-66. (canceled)
